(19) United States
(12) Patent Application Publication
(10) Pub. No.: US 2025/0385008 A1
(43) Pub. Date: Dec. 18, 2025

(54) EMERGENCY PATIENT OBJECTIVE TRIAGE SYSTEM AND METHOD

(71) Applicant: SEOUL NATIONAL UNIVERSITY HOSPTIAL, Seoul (KR)

(72) Inventor: Dong Keon LEE, Gyeonggi-do (KR)

(21) Appl. No.: 18/574,613

(22) PCT Filed: Jun. 28, 2022
(86) PCT No.: PCT/KR2022/009270

§ 371 Date: Nov. 29, 2024

(30) Foreign Application Priority Data
Jun. 28, 2021 (KR) 10-2021-0083997

(51) Int. Cl. G16H 50/30; G06F 18/27; G16H 10/60; G16H 40/20; G16H 50/20

(52) U.S. Cl. CPC G16H 50/30; G06F 18/27; G16H 10/60; G16H 40/20; G16H 50/20

(57) ABSTRACT

Disclosed are an emergency patient objective triage system and method, the system comprising: a basic information unit that obtains data related to basic information of a patient; a visiting information unit that obtains data related to objective visiting information measured in relation to the patient; a symptom unit that distinguishes between subjective information and objective information according to a pre-determined criterion in relation to a main symptom of the patient, and obtains data related to the objective information; and a triage unit that automatically classifies severity of an emergency patient on the basis of at least the data obtained by the visiting information unit from among the pieces of data obtained by the basic information unit, the visiting information unit, and the symptom unit.

### TECHNICAL FIELD

The present invention relates to an emergency patient objective severity classification system and method for quickly classifying severity only with objective information excluding subjective information of a visiting patient in an emergency situation.

### BACKGROUND ART

Triage refers to classifying patients by symptoms to determine priorities for emergency treatment and patient transport. In particular, KTAS (Korean Triage and Acuity Scale, Korean Triage tool for emergency patients) classifies severity of all patients visiting the emergency room based on standardized criteria.

FIG. 1 is a KTAS classification table, which is a system that serves as an existing standard for classifying severity of emergency patients. Referring to FIG. 1, the severity is classified according to its grade into five grades, and the lower the grade number, the treatment priority is higher. The grade is: Emergency grade 1 for patients who need immediate treatment because their lives are at risk; Emergency grade 2 for patients whose lives may be at risk and who require medical attention within the first hour; Emergency grade 3 for patients whose lives are not currently in danger but who need to be treated within a second hour later than the first hour because a serious condition may occur; Emergency grade 4 for patients who are not in critical condition and whose condition is unlikely to worsen even if treated within the third hour, which is later than the second hour; and Emergency grade 5 for non-emergency patients who need to be treated within the fourth hour, which is later than the third hour. Priority medical treatment is determined in emergency medical care depending on the severity, and priority is given to emergency treatment and transport by classifying the patient's mild or severe condition.

However, when there are a large number of patients, it is not easy to classify the severity for each individual patient in an emergency situation because the best medical care needs to be provided to many patients by a limited number of personnel. In addition, even if someone is not a professional personnel, he or she may need to prioritize emergency treatment and transport by distinguishing the patient's mild or severe conditions. Therefore, it is necessary to quickly classify the severity only with objective information excluding subjective information of patients who visits hospital.

### DISCLOSURE

### Technical Problem

The present invention is intended to solve the above-mentioned problems, and provide an emergency patient objective severity classification system and method for quickly classifying severity only with objective information excluding subjective information of a patient in an emergency situation.

### Solution to Problem

Exemplary embodiments of the present invention provides an objective severity classification system for emergency patients, comprising: a basic information unit that acquires data related to a patient's basic information; a hospital visitation information unit that acquires data related to objective hospital visitation information measured about the patient; a symptom unit that distinguishes subjective information and objective information according to predetermined criteria regarding the patient's main symptom and obtains data regarding the objective information; and a severity classification unit that automatically classifies severity of emergency patient based on at least the data obtained from the hospital visitation information unit among the data obtained from the basic information unit, the hospital visitation information unit and the symptom unit.

Further, other exemplary embodiments of the present invention provides an emergency patient objective severity classification method performed by a processor, comprising: a basic information input step for acquiring data related to a patient's basic information; a hospital visitation information input step for acquiring data related to objective hospital visitation information measured about the patient; a symptom input step for distinguishing subjective information and objective information according to predetermined standards regarding the patient's main symptom and obtaining data regarding the objective information; and a severity classification step for automatically classifying severity of emergency patient based on at least the data obtained in the hospital visitation information input step among the data acquired in the basic information input step, hospital visitation information input step and symptom input step.

In one embodiment of the system, the data obtained from the basic information unit comprises at least one of the patient's gender and date of birth.

In one embodiment of the system, the data obtained from the hospital visitation information unit is at least one measurement value of the patient's systolic blood pressure (SBP), diastolic blood pressure (DBP), pulse rate (PR), respiratory rate (RR), body temperature (BT), oxygen saturation (SpO2), and Glasgow Coma Scale (GCS).

In one embodiment of the system, the level of severity is based on the Korean emergency triage tool (KTAS), classified according to grade, and the lower the grade number, the higher the treatment priority, and the grade is classified as follow: Emergency grade 1 for patients who are life-threatening and require immediate medical attention; Emergency grade 2 for patients whose lives may be at risk and who require medical attention within a first hour; Emergency grade 3 for patients whose lives are not currently in danger but who need to be treated within a second hour later than the first hour because a serious condition may occur; Emergency grade 4 for patients who are not in critical condition and whose condition is unlikely to worsen even if treated within a third hour, which is later than the second hour; and Emergency grade 5 for non-emergency patients who need to be treated within a 4th hour, which is later than the 3rd hour.

In one embodiment of the system, based on the systolic blood pressure (SPB) and pulse rate (PR) among the data obtained from the hospital visitation information unit, the severity classification unit classifies the severity of the patient as follows: grade 1 if systolic blood pressure (SPB)<80 mmHg; grade 2 if (pulse rate (PR)>100 beats/min or pulse rate (PR)<60 beats/min) and 80≤ systolic blood pressure (SPB)<100 mmHg; and grade 3 if (pulse rate (PR)>100 beats/min or pulse rate (PR)<60 beats/min) and systolic blood pressure (SPB)≥100 mmHg; and the smaller the number of classified grades, the higher the patient's treatment priority.

In one embodiment of the system, wherein based on the respiratory rate (RR) among the data obtained from the hospital visitation information unit, the severity classification unit classifies the severity of the patient as follows: grade 2 if respiratory rate (RR)≥28 breaths/min or respiratory rate (RR)<14 breaths/min; and grade 3 if 20 breaths/min≤respiratory rate (RR)<27 breaths/min; and the smaller the number of classified grades, the higher the patient's treatment priority.

In one embodiment of the system, based on oxygen saturation (SpO2) among the data obtained from the hospital visitation information unit, the severity classification unit classifies the severity of the patient as follows: grade 1 if oxygen saturation (SpO2)<90%; grade 2 if 90%≤oxygen saturation (SpO2)<92%; and grade 3 if 92%≤oxygen saturation (SpO2)<94%; and the smaller the number of classified grades, the higher the patient's treatment priority.

In one embodiment of the system, based on the Glasgow Coma Scale (GCS) among the data obtained from the above information unit, the severity classification unit classifies the severity of the patient as follows: grade 2 if Glasgow Coma Scale GCS)≤7 points; grade 3 if 7 points<Glasgow Coma Scale (GCS)≤13 points; and the smaller the number of classified grades, the higher the patient's treatment priority.

In one embodiment of the system, the severity classification unit classifies the severity of the patient based on systemic inflammatory response syndrome (SIRS) response criteria based on pulse rate (PR), respiratory rate (RR), and body temperature (BT) among the data obtained from the hospital visitation information unit, wherein the systemic inflammatory response syndrome (SIRS) satisfies two or more of i) pulse rate (PR)>90 beats/min, ii) respiratory rate (RR)>20 beats/min, iii) body temperature (BT)>38° C. or body temperature (BT)<36° C., wherein the severity level is classified as follows: step 2 if all three SIRS satisfaction criteria are met; step 3 if two of the above SIRS satisfaction criteria are met; and step 4 if only body temperature (BT)>38° C. or body temperature (BT)<36° C. is satisfied among the above SIRS satisfaction criteria; and the smaller the number of classified grades, the higher the patient's treatment priority.

In one embodiment of the system, when the severity classification unit classifies the severity based on multiple items among the data acquired from the hospital visitation information unit, if the classification grades corresponding to each of the multiple items are different, the highest grade in the patient's treatment priority with smallest number among the multiple grades is selected as the final grade.

In one embodiment of the system, the severity classification unit presents classification results appropriate for the patient's age based on the patient's date of birth among the data obtained from the basic information unit.

In one embodiment of the system, if the patient is an elderly patient according to preset standards, the severity classification unit raises the severity classification grade of the patient by one grade.

In one embodiment of the system, the severity classification unit make a final classification through logistic regression, which reflects the patient's age as a continuous variable.

### Advantageous Effects of the Invention

According to the emergency patient objective severity classification system and method of the present invention, it is possible to respond according to the patient's severity condition by classifying patients per their severity only with objective information in an emergency situation, and also to expect the efficiency and objectification of the emergency medical system.

The effects of the present invention are not limited to the effects mentioned above, and other effects not mentioned will be clearly understood by those skilled in the art from the description of the claims.

### BRIEF DESCRIPTION OF DRAWINGS

In order to more clearly explain the technical solutions of the embodiments of the present invention or the prior art, drawings necessary for the description of the embodiments are briefly introduced below. It should be understood that the drawings below are for illustrative purposes only and not for limiting purposes of the embodiments of the present specification. Additionally, for clarity of explanation, some elements may be shown in the drawings below with various modifications, such as exaggeration, omission, etc.

FIG. 1 is the KTAS classification table which serves as an existing standardized system for classifying the severity of emergency patients.

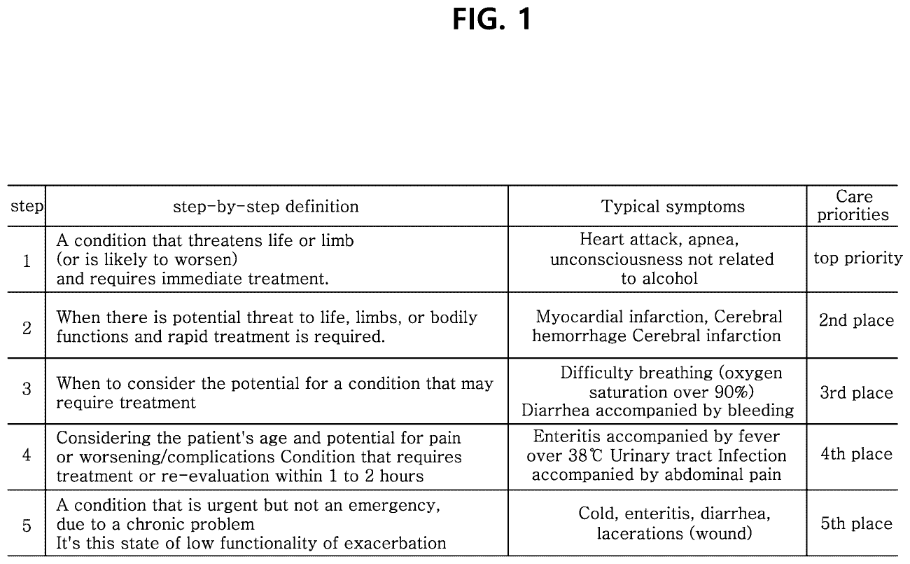

FIG. 2 is a schematic diagram of an emergency patient objective severity classification system according to an embodiment of the present invention.

FIG. 3 is a flowchart illustrating a method for objectively classifying the severity of an emergency patient according to an embodiment of the present invention.

FIG. 4 is an image in which the severity classification unit automatically classifies the severity of an emergency patient based on data obtained by the hospital visitation information unit according to an embodiment of the present invention.

FIG. 5 is an image showing a process of finally classifying the severity level of an emergency patient by the severity classification unit according to an embodiment of the present invention.

### MODE FOR INVENTION

The terminology used herein is only intended to refer to specific embodiments and is not intended to limit the invention. As used herein, singular forms include plural forms unless clearly indicated otherwise. As used in the specification, the meaning of “comprising” specifies a particular characteristic, area, integer, step, operation, element and/or ingredient, and does not exclude the presence or addition of another characteristic, area, integer, step, operation, element and/or ingredient.

Although not defined differently, all terms used herein, including technical and scientific terms, have the same meaning as those generally understood by those skilled in the art in the technical field to which the present invention pertains. Terms defined in commonly used dictionaries are further interpreted as having meanings consistent with related technical literature and currently disclosed content, and are not interpreted in ideal or very formal meanings unless defined.

Embodiments of the present invention will be described below in detail with reference to the drawings.

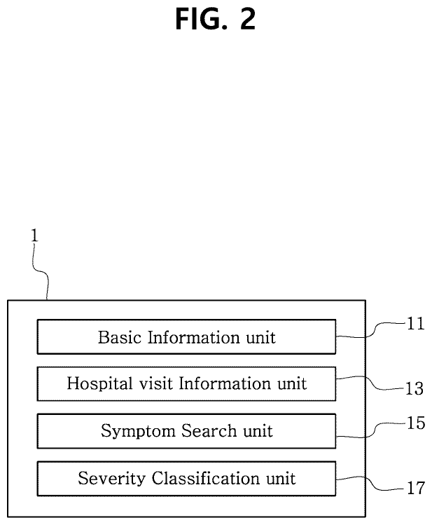

FIG. 2 is a schematic diagram of an emergency patient objective severity classification system according to an embodiment of the present invention.

Referring to FIG. 2, the emergency patient objective severity classification system 1 may include a basic information unit 11, a hospital visitation information unit 13, a symptom unit 15, and a severity classification unit 17.

The basic information unit 11 acquires data related to basic information of a patient. The data may be at least one of a patient's gender and a date of birth. The gender may be selected by the user by clicking among men/women. The age may be based on 15 years of age, and 15 years old or older adults may be targeted, but is not limited thereto.

The hospital visitation information unit 13 acquires data related to objective hospital visitation information excluding subjective information of a patient. The data may be measured values of at least one of systolic blood pressure (SBP), diastolic blood pressure (DBP), pulse rate (PR), respiratory rate (RR), body temperature (BT), oxygen saturation (SpO2), and Glasgow Coma Scale (GCS) of the patient. When the user clicks the measurement value input box on the display, the keypad may be activated to input a numerical value. The data acquired by the hospital visitation information unit can be used to automatically classify the KTAS grade as the vital signs primary consideration. In the case of body temperature, it may be input to the first decimal place, but is not limited thereto. Even if all of the plurality of measurement values are not input, only the input measurement value may be used for the vital signs primary consideration.

The symptom unit 15 divides subjective information and objective information according to predetermined criteria regarding the patient's main symptoms and acquires data on objective information. The data obtained by the symptom unit 15 is an objective information related to one or more main symptoms of the patient, and the symptom unit may be configured to receive the patient's symptoms as a search term, search for a plurality of main symptoms related to the input search term, and present to make user select at least one main symptom among a plurality of searched main symptoms. A plurality of symptoms may be input to the main symptom, and the selected main symptom may be corrected by adding or deleting.

The objective information for each symptom acquired by the symptom unit 15 includes a plurality of items. In an embodiment, the objective information for each symptom is an objective information in terms of vital signs related to the vital signs primary considerations obtained by the hospital visitation information unit. For example, the symptom item may include an information related to the level of consciousness, hemodynamic state, respiratory state and/or body temperature state. Additionally, symptom items may include an information related to bleeding conditions, accident mechanisms (details), acute or chronic central pain, and acute or chronic peripheral pain.

The severity classification unit 17 automatically classifies the severity of emergency patients based on at least data obtained from the hospital visitation information unit among the data obtained from the basic information unit 11, the hospital visitation information unit 13, and the symptom unit 15. The severity classification unit 17 may classify the severity by applying the value of each input item to a preset rule. In an embodiment, the severity may be based on a Korean emergency patient classification tool (KTAS).

In one embodiment, based on systolic blood pressure (SPB) and pulse rate (PR) among the data obtained by the above hospital visitation information, the severity classification unit can be classified into three grades: grade 1 if systolic blood pressure (SPB)<80 mmHg; grade 2 if (pulse rate (PR)>100 beats/min or pulse rate (PR)<60 beats/min) and 80≤systolic blood pressure (SPB)<100 mmHg; and grade 3 if (pulse rate (PR)>100 beats/min or pulse rate (PR)<60 beats/min) and systolic blood pressure (SPB)≥100 mmHg.

In one embodiment, based on the respiratory rate (RR) among the data obtained by the hospital visitation information unit, the severity classification unit may classify the severity of the patient into: grade 2 for respiratory rate (RR)≥28 times/min or respiratory rate (RR)<14 times/min; and grade 3 for 20 times/min≤respiratory rate (RR)<27 times/min.

In one embodiment, based on the oxygen saturation (SpO2) among the data obtained by the above hospital visitation information unit, the severity classification unit can classify the severity of the patient into: grade 1 if oxygen saturation (SpO2)<90%; grade 2 if 90%≤oxygen saturation (SpO2)<92%; and grade 3 if 92%≤oxygen saturation (SpO2)<94%.

In one embodiment, based on the Glasgow Coma Scale (GCS) among the data obtained from the above information unit, the severity classification unit classifies the severity of the patient as follows: grade 2 if Glasgow Coma Scale (GCS)≤7 points; grade 3 if 7 points<Glasgow Coma Scale (GCS)≤13 points.

In one embodiment of the system, the severity classification unit classifies the severity of the patient based on systemic inflammatory response syndrome (SIRS) response criteria based on pulse rate (PR), respiratory rate (RR), and body temperature (BT) among the data obtained from the hospital visitation information unit, herein the systemic inflammatory response syndrome (SIRS) satisfies two or more of i) pulse rate (PR)>90 beats/min, ii) respiratory rate (RR)>20 beats/min, iii) body temperature (BT)>38° C. or body temperature (BT)<36° C., wherein the severity level is classified as follows: step 2 if all three SIRS satisfaction criteria are met; step 3 if two of the above SIRS satisfaction criteria are met; and step 4 if only body temperature (BT)>3820 C. or body temperature (BT)<36° C. is satisfied among the above SIRS satisfaction criteria. When the severity classification unit 17 classifies the severity based on a plurality of items among the data acquired by the hospital visitation information unit 13, if the classification grades corresponding to each of the plurality of items are different, the grade with the highest treatment priority of the patient having the lowest number in the multiple grades may be selected as the final grade.

The severity classification unit 17 may present a classification result suitable for the patient's age based on the patient's birth date among the data acquired by the basic information unit 11. In an embodiment, when the patient is an elderly patient according to a preset criterion, the severity classification unit may classify the patient by upgrading the severity classification grade of the patient by one grade. Statutory criteria for classifying elderly patients may be used as the above criteria, and patients over 65 years of age may be classified as elderly patients. The statutory criteria may be, for example, a legal standard used in the Elderly Outpatient Fixed Payment System and the Elderly Welfare Act.

The severity classification unit 17 may make a final classification through logistic regression reflecting the patient's age as a continuous variable. For example, the severity classification unit can classify the severity classification by gradually increasing the severity level as the patient's age increases.

The method for objectively classifying the severity of an emergency patient according to another aspect of the present invention is performed by a computing device including a processor. The computing device including the processor may be performed, for example, by the emergency patient objective severity classification system 1 or some components (e.g., basic information unit 11, hospital visitation information unit 13, symptom unit 15, and severity classification unit 17), or by other computing devices. Hereinafter, for clarity of explanation, the present invention will be described in more detail with embodiments performed by the emergency patient objective severity classification system 1.

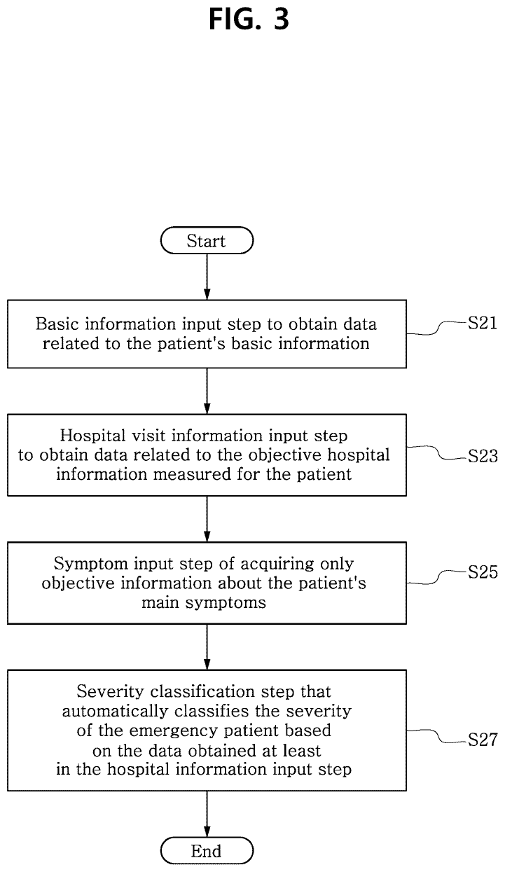

FIG. 3 is a flowchart of a method for objectively classifying the severity of an emergency patient according to an embodiment of the present invention.

Referring to FIG. 3, the method for objective severity classification of emergency patients is: a basic information input step (S21) for acquiring data related to basic information of patients (e.g., by basic information unit 11); hospital visitation information input step (S23) for acquiring data related to objective information measured for patients (e.g., by hospital visitation information unit 13); symptom input step for classifying subjective and objective information according to predetermined criteria and obtaining data on objective information (e.g., by symptom unit 15); and a severity classification step (S27) for automatically classifying severity of emergency patient based on at least the data obtained in the hospital visitation information input step among the data acquired in the basic information input step, hospital visitation information input step and symptom input step (e.g., by severity classification unit 17).

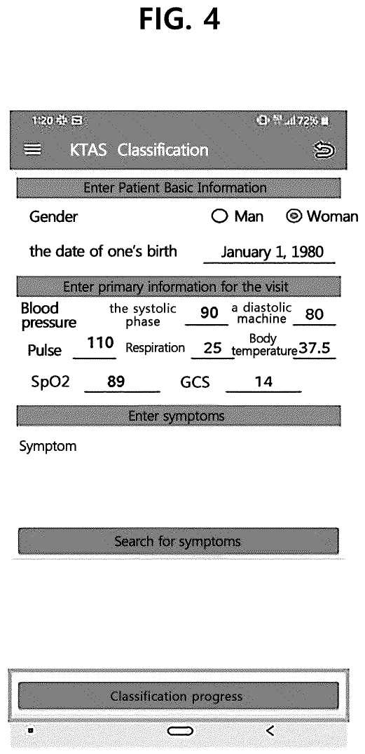

FIG. 4 is an image in which the severity classification unit automatically classifies the severity of an emergency patient based on data obtained by the hospital visitation information unit according to an embodiment of the present invention.

Referring to FIG. 4, the systolic blood pressure (SPB) is 90 mmHg and the pulse rate (PR) is 110 times/min among the data obtained by the hospital visitation information unit. Based on this, the severity classification unit may classify the severity of the patient as grade 2 because this is the case of (pulse rate (PR)>100 times/min or pulse rate (PR)<60 times/min) and 80≤systolic blood pressure (SPB)<100 mmHg. Based on the respiratory rate (RR) of 25 times/min among the data obtained by the hospital visitation information unit, the severity classification unit may classify the severity of the patient into grade 3 as this is the case of 20 times/min≤respiratory rate (RR)<27 times/min. Based on the oxygen saturation (SpO2) of 89% among the data obtained by the hospital visitation information unit, the severity classification unit may classify the severity of the patient into grade 1 as this is the case of oxygen saturation (SpO2)<90%. Based on that the Glasgow Coma Scale (GCS) among the data obtained by the hospital visitation information unit is 14 points, the severity classification unit may classify the severity of the patient into grade 4 or higher. Based on pulse rate (PR) 110, respiratory rate (RR) 25, and body temperature (BT)37.5° C. among the data obtained by the hospital visitation information unit, i) and ii) are met among i) pulse rate (PR)>90 times/min, ii) respiratory rate (RR)>20 times/min, ii) body temperature (BT) >38° C. or body temperature (BT)<36° C., the severity classification unit may classify the patient's severity into grade 3.

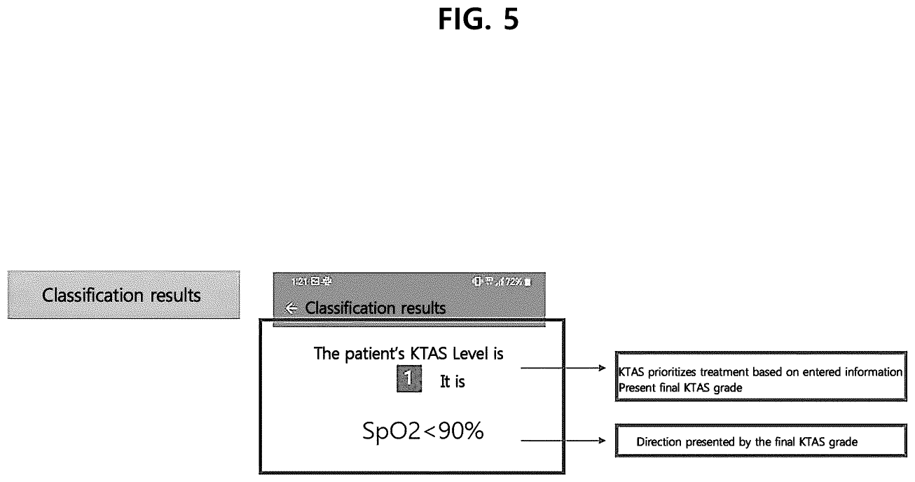

FIG. 5 is an image showing a process of finally classifying the severity grade of an emergency patient by the severity classification unit according to an embodiment of the present invention.

Referring to FIG. 5, when the severity classification unit classifies the severity based on multiple items among the data acquired by the hospital visitation information unit, if the classification grades corresponding to each of the multiple items are different, the lowest number grade of highest treatment priority among the multiple grades may be selected as the final grade. Referring back to FIG. 4, multiple grades exist according to each input value. For example, grade 2 for systolic blood pressure (SPB) 90 mmHg and pulse rate (PR) 110 input values, grade 3 for respiratory rate (RR) 25 times/min, grade 1 for oxygen saturation (SpO2) 89% input values, grade 3 according to the System Inflammatory Response Syndrome (SIRS) response criteria. The severity classification unit 17 may classify the grade 1 having the lowest number among the plurality of grades (grade 2, grade 3, grade 1 and grade 3) as the final grade, and provides a direction for treatment by presenting the items that became a cause of the grade classification.

Referring to FIG. 5, oxygen saturation (SpO2)<90%, which is the cause classified as the first grade, is also presented. In an embodiment, KTAS grades classified for each input item may be listed together with the presentation of the final classification grade. By showing different KTAS grades results according to the input information, the user may compare each grade at a glance, thereby providing convenience.

According to this emergency patient objective severity classification system and method, patients are classified by severity only with objective information in an emergency situation, so it is possible to respond according to the patient's severity condition by classifying patients per their severity only with objective information in an emergency situation, and also to expect the efficiency and objectification of the emergency medical system.

The operation of the emergency patient objective severity classification method according to the above-described embodiments may be at least partially implemented as a computer program and recorded on a computer-readable recording medium. For example, it may be implemented with a program product consisting of a computer-readable medium comprising program code, which may be executed by a processor for performing any or all of the steps, operations, or processes described above.

The method of objectively classifying the severity of an emergency patient according to another aspect of the present invention may be performed by a computing device including a processor. The computing device may be a computing device such as a desktop computer, a laptop computer, a laptop computer, a smart phone, or the like, or any device that may be integrated. A computer is a device having one or more alternative and special purpose processors, memory, storage space, and networking components (e.g., either wireless or wired). The computer may run, for example, an operating system compatible with Microsoft's Windows, Apple OS X or iOS, Linux distribution, or an operating system such as Google's Android OS.

The computer-readable recording medium includes all types of recording identification devices in which data readable by a computer is stored. Examples of computer-readable recording media include ROM, RAM, CD-ROM, magnetic tape, floppy disk, optical data storage identity verification device, and the like. In addition, computer-readable recording media may be distributed to network-connected computer systems, and computer-readable codes may be stored and executed in a distributed manner. In addition, functional programs, codes, and code segments for implementing this embodiment will be easily understood by those skilled in the art to which this embodiment belongs.

The present invention discussed above has been described with reference to the embodiments shown in the drawings, but this is only exemplary, and anyone with ordinary skill in the field will understand that various modifications and modifications of the embodiments are possible. However, such modifications should be considered to be within the scope of the technical protection of the present invention. Therefore, the true technical protection scope of the present invention should be determined by the technical spirit of the attached patent claims.

### INDUSTRIAL APPLICABILITY

The present invention relates to an emergency patient objective severity classification system and method for quickly classifying severity only with objective information excluding subjective information of a patient in an emergency situation. Accordingly, it is possible to respond according to the patient's severity condition by classifying patients per their severity only with objective information in an emergency situation, and also to expect the efficiency and objectification of the emergency medical system.

1. An objective severity classification system for emergency patients, comprising:
a basic information unit that acquires data related to a patient's basic information;
a hospital visitation information unit that acquires data related to objective hospital visitation information measured about the patient;
a symptom unit that distinguishes subjective information and objective information according to predetermined criteria regarding the patient's main symptom and obtains data regarding the objective information; and
a severity classification unit that automatically classifies severity of emergency patient based on at least the data obtained from the hospital visitation information unit among the data obtained from the basic information unit, the hospital visitation information unit and the symptom unit.

2. The system according to claims 1, wherein the data obtained from the basic information unit comprises at least one of the patient's gender and date of birth.

3. The system according to claims 1, wherein the data obtained from the hospital visitation information unit is at least one measurement value of the patient's systolic blood pressure (SBP), diastolic blood pressure (DBP), pulse rate (PR), respiratory rate (RR), body temperature (BT), oxygen saturation (SpO2), and Glasgow Coma Scale (GCS).

4. The system according to claims 1, wherein the level of severity is based on the Korean emergency triage tool (KTAS), classified according to grade, and the lower the grade number, the higher the treatment priority, and the grade is classified as follow:
Emergency grade 1 for patients who are life-threatening and require immediate medical attention;
Emergency grade 2 for patients whose lives may be at risk and who require medical attention within a first hour;
Emergency grade 3 for patients whose lives are not currently in danger but who need to be treated within a second hour later than the first hour because a serious condition may occur;
Emergency grade 4 for patients who are not in critical condition and whose condition is unlikely to worsen even if treated within a third hour, which is later than the second hour; and
Emergency grade 5 for non-emergency patients who need to be treated within a 4th hour, which is later than the 3rd hour.

5. The system according to claims 3, wherein based on the systolic blood pressure (SPB) and pulse rate (PR) among the data obtained from the hospital visitation information unit, the severity classification unit classifies the severity of the patient as follows:
grade 1 if systolic blood pressure (SPB)<80 mmHg;
grade 2 if (pulse rate (PR)>100 beats/min or pulse rate (PR)<60 beats/min) and 80≤systolic blood pressure (SPB)<100 mmHg; and
grade 3 if (pulse rate (PR)>100 beats/min or pulse rate (PR)<60 beats/min) and systolic blood pressure (SPB)≥100 mmHg; and
the smaller the number of classified grades, the higher the patient's treatment priority.

6. The system according to claims 3, wherein based on the respiratory rate (RR) among the data obtained from the hospital visitation information unit, the severity classification unit classifies the severity of the patient as follows:
grade 2 if respiratory rate (RR)≥28 breaths/min or respiratory rate (RR)<14 breaths/min; and
grade 3 if 20 breaths/min≤respiratory rate (RR)<27 breaths/min; and
the smaller the number of classified grades, the higher the patient's treatment priority.

7. The system according to claims 3, wherein based on oxygen saturation (SpO2) among the data obtained from the hospital visitation information unit, the severity classification unit classifies the severity of the patient as follows:
grade 1 if oxygen saturation (SpO2)<90%;
grade 2 if 90%≤oxygen saturation (SpO2)<92%; and
grade 3 if 92%≤oxygen saturation (SpO2)<94%; and
the smaller the number of classified grades, the higher the patient's treatment priority.

8. The system according to claims 3, wherein based on the Glasgow Coma Scale (GCS) among the data obtained from the above information unit, the severity classification unit classifies the severity of the patient as follows:
grade 2 if Glasgow Coma Scale (GCS)≤7 points; and
grade 3 if 7 points<Glasgow Coma Scale (GCS)≤13 points; and
the smaller the number of classified grades, the higher the patient's treatment priority.

9. The system according to claims 3, wherein the severity classification unit classifies the severity of the patient based on systemic inflammatory response syndrome (SIRS) response criteria based on pulse rate (PR), respiratory rate (RR), and body temperature (BT) among the data obtained from the hospital visitation information unit, wherein the systemic inflammatory response syndrome (SIRS) satisfies two or more of i) pulse rate (PR)>90 beats/min, ii) respiratory rate (RR)>20 beats/min, iii) body temperature (BT) >38° C. or body temperature (BT)<36° C., wherein the severity level is classified as follows:
step 2 if all three SIRS satisfaction criteria are met;
step 3 if two of the above SIRS satisfaction criteria are met; and
step 4 if only body temperature (BT)>38° C. or body temperature (BT)<36° C. is satisfied among the above SIRS satisfaction criteria; and
the smaller the number of classified grades, the higher the patient's treatment priority.

10. The system according to claims 3, wherein when the severity classification unit classifies the severity based on multiple items among the data acquired from the hospital visitation information unit, if the classification grades corresponding to each of the multiple items are different, the highest grade in the patient's treatment priority with smallest number among the multiple grades is selected as the final grade.

11. The system according to claims 2, wherein the severity classification unit presents classification results appropriate for the patient's age based on the patient's date of birth among the data obtained from the basic information unit.

12. The system according to claims 11, wherein if the patient is an elderly patient according to preset standards, the severity classification unit raises the severity classification grade of the patient by one grade.

13. The system according to claims 11, wherein the severity classification unit make a final classification through logistic regression, which reflects the patient's age as a continuous variable.

14. An emergency patient objective severity classification method performed by a processor, comprising:
a basic information input step for acquiring data related to a patient's basic information;
a hospital visitation information input step for acquiring data related to objective hospital visitation information measured about the patient;
a symptom input step for distinguishing subjective information and objective information according to predetermined standards regarding the patient's main symptom and obtaining data regarding the objective information; and
a severity classification step for automatically classifying severity of emergency patient based on at least the data obtained in the hospital visitation information input step among the data acquired in the basic information input step, hospital visitation information input step and symptom input step.

15. A computer-readable recording medium that is readable by a computer and stores program instructions operable by the computer, wherein when the program instructions are executed by a processor of the computer, the processor determines the objective severity of an emergency patient according to claim 14.
